(19) United States
(12) Patent Application Publication
(10) Pub. No.: US 2018/0055370 A1
(43) Pub. Date: Mar. 1, 2018

(54) PHOTOACOUSTIC APPARATUS

(71) Applicant: CANON KABUSHIKI KAISHA, Tokyo (JP)

(72) Inventors: Atsushi Takahashi, Ebina-shi (JP); Tatsuki Fukui, Yokohama-shi (JP); Satoshi Ogawa, Yokohama-shi (JP)

(21) Appl. No.: 15/553,228

(22) PCT Filed: Mar. 18, 2016
(86) PCT No.: PCT/JP2016/059787

§ 371 Date: Aug. 24, 2017

(30) Foreign Application Priority Data
Mar. 26, 2015 (JP) 2015-064323

(51) Int. Cl. A61B 5/00; A61B 5/15; A61M 5/00

(52) U.S. Cl. CPC A61B 5/0095; A61B 5/7246; A61B 2576/02; A61M 5/007; A61B 5/150083

(57) ABSTRACT

A photoacoustic apparatus is used that has: a detecting unit that detects photoacoustic waves generated by an object containing contrast agent in first blood vessels of circulating blood and in second blood vessels; and a signal processing unit that generates contrast agent distribution and acquires contrast agent concentration change in the circulating blood. The signal processing unit acquires the position of the first blood vessels on the basis of a time-series change of the contrast agent distribution and the contrast agent concentration change, and lowers the concentration at the position of the first blood vessels on the basis of the contrast agent distribution.

### TECHNICAL FIELD

The present invention relates to a photoacoustic apparatus.

### BACKGROUND ART

Photoacoustic tomography (PAT) has been developed in the medical field as a technology for imaging objects, for instance a living body or the like, using light. In a photoacoustic apparatus that relies on photoacoustic tomography, a living body is irradiated with light from a light source, and there are detected acoustic waves generated by biological tissue, which absorbs energy of pulsed light that propagates and diffuses within the living body. Obtained signals are then subjected to a mathematical analysis process (image reconstruction process), to visualize information associated with an optical characteristic value of the interior of the object. As a result, an initial sound pressure, optical characteristic value, as well as distributions thereof, can be acquired and used, for instance, to identify the distribution of an absorber or the site of a malignant tumor within the living body.

In the PAT, an initial sound pressure P₀ of acoustic waves generated by a light absorber within an object is given by Expression (A) below.

P₀=FΓ·μa·Φ (A)

In the expression, Γ is the Gruneisen parameter, i.e. the quotient of the product of a coefficient of volumetric expansion β and the square of the speed of sound c, divided by the specific heat at constant pressure Cp. Herein, Γ takes on a substantially constant value for a determined object. Further, μa is the absorption coefficient of the light absorber, and Φ, referred to as light fluence, is the amount of light at the position of the light absorber, i.e. the amount of light irradiated to the light absorber.

The photoacoustic apparatus detects the initial sound pressure P₀ of acoustic waves that are generated by the light absorber within the object and that propagate up to the object surface. The initial sound pressure distribution P₀ can be calculated by measuring the change of sound pressure over time and by using an image reconstruction method such as back projection. The distribution of the product of μa and Φ, i.e. a light energy density distribution, is obtained by dividing the initial sound pressure distribution P₀ by the Gruneisen coefficient Γ. The absorption coefficient distribution μa is obtained by dividing the light energy density distribution by the light amount distribution Φ within the object, provided that the light amount distribution Φ has been worked out in some manner.

In a case where photoacoustic tomography is used in living bodies, the spatial distribution of blood can be imaged by exploiting the fact that near-infrared light is absorbed well by hemoglobin in blood. Further, an oxygen saturation distribution can be imaged on the basis of a presence fraction of oxi-deoxyhemoglobin, using light of a plurality of wavelengths. The above principle has been used to develop applications for imaging blood vessels in small animals, and in diagnosis of breast cancer, prostate cancer, carotid artery plaque and the like.

Acoustic waves corresponding to the abundance of a contrast agent can be detected upon administration, as the contrast agent, of an absorber the optical characteristic whereof is known. In the case, for instance, of tumor imaging, attempts have therefore been made towards enhancing the precision of characteristic information by improving image contrast through the use of contrast agents that have the property of collecting at tumor sites.

Herein, Japanese Patent Application Publication No. 2012-527324 (Patent Literature 1) discloses a technology for visualizing blood flow parameters using a contrast agent, by exploiting differences in blood flow parameters between tumor tissue and normal tissue. Generally, new blood vessels are formed extensively in tumor tissue, in order for the tumor tissue to actively receive the supply of nutrients and oxygen from the surroundings. By contrast, the vascular structure of new blood vessels in tumor tissues, for instance pericytes, is immature. It is determined that, as a result, substances permeate more readily in new blood vessels (i.e. blood vessel transmissivity is higher) than in normal blood vessels through which circulating blood flows. This characteristic is referred to as EPR (Enhanced Permeability and Retention). In the present description, blood vessels of circulating blood, which are conceptually opposed to new blood vessels, denote normal blood vessels through which blood circulates to biological tissues. The vascular structure in blood vessels of circulating blood is mature, and is ordinarily thicker than that of new blood vessels. The vascular structure in capillaries and the like, though, may in some instances be thinner than new blood vessels.

In photoacoustic tomography targeted at hemoglobin, however, new blood vessels have a small blood vessel size and exhibit unstable blood flow, and accordingly image contrast may in some instances be insufficient. In this case, the contrast agent is administered externally to the object so that as much contrast agent as possible reaches the tumor tissue, to enhance as a result the image contrast of new blood vessels. Ordinarily, the contrast agent is administered into the blood via a vein or the like, circulates together with the circulating blood into the interior of the body, and reaches thereafter the tumor tissue that includes a neovascular region. Therefore, the contrast agent concentration in the circulation influences significantly the amount of contrast agent that reaches the tumor.

### CITATION LIST

### Patent Literature

PTL 1: Japanese Translation of PCT Application No. 2012-527324

### SUMMARY OF INVENTION

### Technical Problem

If, in photoacoustic tomography using a contrast agent, the contrast agent is present not only in new blood vessels but also in the circulating blood, the contrast agent in the latter case is imaged as well, and hence the image of the neovascular region may in some instances be unclear. This phenomenon is prominent in a case where a low-molecular material, such as indocyanine green (ICG), is used as the contrast agent. Specifically, ICG has a half life of about 3 minutes in blood. When imaging thus a tumor portion using a contrast agent having such a fast elimination rate in blood, the photoacoustic measurement must be performed at a point in time at which the concentration of the contrast agent immediately after administration is still high. Signals from the tumor portion may be however hidden on account of the sizeable presence of contrast agent also in the circulating blood that flows through normal blood vessels at this timing. In a case in particular where the photoacoustic signal intensity is displayed according to a MIP (Maximum Intensity Projection) scheme, which is a maximum value projection method, the transmissivity of the blood vessels is not displayed accurately, and the visibility of the image is impaired.

If a contrast agent having high retention in blood is used, on the other hand, sufficient contrast can be obtained also in tumor portions, since the contrast agent can remain in the tumor tissue by virtue of the EPR effect. Instances of molecular design for imparting a contrast agent with retention in circulating blood include methods that involve controlling material physical properties such as the size, surface charge and so forth of the contrast agent. Specific examples include, for instance, serum-derived proteins such as albumin and IgG, as well as water-soluble synthetic polymers such as polyethylene glycol (material of higher molecular weight than ICG alone) By using these molecules as carriers of the contrast agent, the concentration in the circulating blood of the contrast agent that has been administered can be expected to be secured for a given time or longer, without the contrast agent being trapped in excretory organs such as the kidneys and the liver.

In this method, however, the wait time from contrast agent administration until the measurement starts may be prolonged, by several days in some instances. When using contrast agents having high blood retention, therefore, methods have been sought that allow obtaining high-contrast images in shorter times, from the viewpoint of cost and convenience.

The present invention was arrived at in the light of the above problems. It is an object of the present invention to provide a technology for acquiring images of surrounding tissue, for instance a tumor, with good contrast, in photoacoustic tomography where a contrast agent is used.

### Solution to Problem

The present invention provides a photoacoustic apparatus, comprising:

a light source;

a detecting unit that detects photoacoustic waves generated upon irradiation of light, from the light source, onto an object that has first blood vessels in which circulating blood circulates and second blood vessels having a structure different from that of the first blood vessels, the object containing a contrast agent in the first and second blood vessels; and

a signal processing unit that generates contrast agent distribution information by working out a concentration of the contrast agent in each unit region within the object using the photoacoustic waves, and that acquires contrast agent concentration change information denoting the change with time of the concentration of the contrast agent in the circulating blood

wherein the signal processing unit:

generates the contrast agent distribution information a plurality of times in response to a plurality of light irradiations from the light source;

acquires the position of the first blood vessels on the basis of a time-series change of the contrast agent distribution information having been generated a plurality of times, and the contrast agent concentration change information; and

performs correction of lowering the concentration of the contrast agent at the position of the first blood vessels on the basis of the contrast agent distribution information.

The present invention also provides photoacoustic apparatus, comprising:

a light source;

a detecting unit that detects photoacoustic waves generated upon irradiation of light, from the light source, onto an object that has first blood vessels in which circulating blood circulates and second blood vessels having a structure different from that of the first blood vessels, the object containing a contrast agent in the first and second blood vessels; and

a signal processing unit that generates light absorber distribution information by working out a concentration of a light absorber for each unit region within the object using the photoacoustic waves, generates contrast agent distribution information within the object using the light absorber distribution information, and acquires contrast agent concentration change information that denotes a change with time of the concentration of the contrast agent in the circulating blood,

wherein the signal processing unit:

generates the contrast agent distribution information a plurality of times in response to a plurality of light irradiations from the light source;

acquires the position of the first blood vessels on the basis of a time-series change of the contrast agent distribution information having been generated a plurality of times, and the contrast agent concentration change information; and

performs correction of lowering the concentration of the light absorber at the position of the first blood vessels, on the basis of the light absorber distribution information.

### Advantageous Effects of Invention

The present invention allows acquiring images of surrounding tissue, for instance a tumor, with good contrast, in photoacoustic tomography where a contrast agent is used.

Further features of the present invention will become apparent from the following description of exemplary embodiments with reference to the attached drawings.

### BRIEF DESCRIPTION OF DRAWINGS

FIG. 1 is a diagram illustrating an object information acquisition device according to an embodiment;

FIG. 2 is a diagram illustrating an example of a time-series change of the concentration of contrast agent for ICG-PEG (20 k);

FIG. 3 is a diagram illustrating a flow of object image acquisition according to an embodiment;

FIG. 4 is another diagram illustrating an object information acquisition device according to an embodiment; and

FIG. 5A to FIG. 5D are diagrams illustrating a comparison technique of information A and information B.

### DESCRIPTION OF EMBODIMENTS

Preferred embodiments of the present invention will be explained next with reference to accompanying drawings. The dimensions, materials, and shapes of constituent parts, relative positions between the constituent parts, and other features described below are to be modified, as appropriate, in accordance with the configuration of the equipment to which the present invention is to be applied, and in accordance with various other conditions, and therefore do not constitute features that limit the scope of the invention to the disclosure that follows hereafter.

The present invention relates to a technology for detecting acoustic waves that propagate from an object, and for generating and acquiring characteristic information about the interior of the object. Accordingly, the present invention can be regarded as an object information acquisition device or control method thereof, or as an object information acquisition method or signal processing method. The present invention can further be viewed as a program for causing the foregoing methods to be executed in data processing device provided with hardware resources, such as a CPU and the like, and as a storage medium in which such a program is stored.

The object information acquisition device of the present invention encompasses devices that rely on the photoacoustic tomography technology, which involves irradiating an object with light (electromagnetic waves), and receiving (detecting) propagating acoustic waves that are generated at specific positions inside the object, or at the object surface, on account of the photoacoustic effect. Such object information acquisition devices obtain, for instance in the form of image data, characteristic information of the interior of the object on the basis of photoacoustic measurements, and, accordingly, are also referred to as photoacoustic imaging devices.

Characteristic information in a photoacoustic apparatus denotes herein the source distribution of acoustic waves that are generated, and an initial sound pressure distribution within the object, resulting from irradiation of light, or a light energy absorption density distribution or absorption coefficient distribution, or concentration distribution of constituent substances in tissues, derived from the initial sound pressure distribution. Specifically, characteristic information includes, for instance, blood component distributions such as oxi-deoxyhemoglobin concentration distributions and an oxygen saturation distribution worked out from the foregoing, and distributions of fat, collagen, water and the like. The characteristic information may be worked out not in the form of numerical value data but in the form of distribution information on each position of within the object. Specifically, distribution information such as an absorption coefficient distribution, an oxygen saturation distribution or the like may be used as object information. The characteristic information derived from photoacoustic waves is also referred to as function information that exhibits functional differences arising from substances within the object.

As used in the present invention, the term acoustic wave typically refers to ultrasonic waves, and encompasses elastic waves referred to as sound waves and acoustic waves. Acoustic waves generated on account of the photoacoustic effect are referred to as photoacoustic waves or photo-ultrasonic waves. Electrical signals resulting from conversion of acoustic waves by a probe or the like are also referred to as acoustic signals.

The main object of the device of the present invention includes, for instance, diagnosis of malignant tumors, vascular diseases and the like in humans and animals, as well as follow up in chemotherapy. Accordingly, conceivable objects include various biological segments (breasts, fingers, hands, feet and the like) in human bodies and animals. The device creates an image of a light absorber that is present in the interior of the object (for instance, oxi-deoxyhemoglobin in blood, blood vessels comprising a large amount of blood, or artificially introduced contrast agents), or of light absorbers (coloring materials such as melanin) on the object surface.

### Embodiment 1

(Subject Information Acquisition Device)

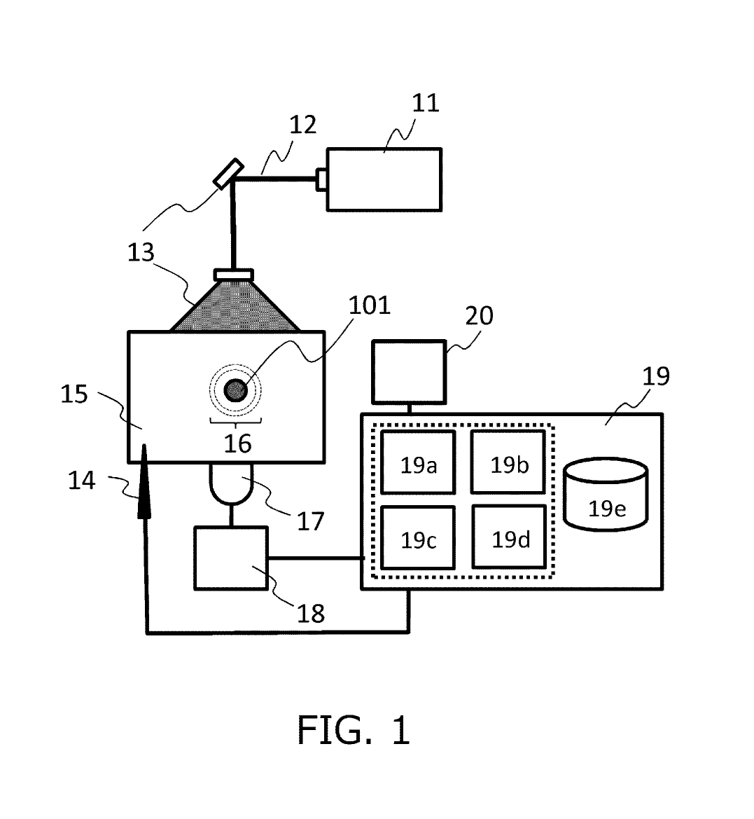

A device configuration according to the present embodiment will be explained next with reference to FIG. 1. The device has a light source 11, an optical system 13, a contrast agent administering unit 14, an acoustic wave detecting unit 17, a signal collecting unit 18, a signal processing unit 19 and a display device 20. In a basic process flow, firstly light 12 emitted by the light source 11 passes through the optical system 13 and the object 15 is irradiated with the light 12. The object 15 contains a contrast agent that is administered by the contrast agent administering unit 14. The acoustic wave detecting unit 17 detects photoacoustic waves 16 generated by a light absorber 101 such as the contrast agent, and converts the photoacoustic waves 16 to an electrical signal. The electrical signal is converted to characteristic information by being processed in the signal collecting unit 18, the signal processing unit 19 and so forth, and the conversion result is displayed on the display device 20.

(Method for Acquiring Time-Series Contrast Agent Concentration Change Information)

In the present invention the “information A” and “information B” described below are chronologically acquired, in the form of a time series, from the object to which the contrast agent has been administered. Firstly, information A is the change of the contrast agent concentration in circulating blood. The term circulating blood denotes blood in normal blood vessels through which blood circulates to ordinary biological tissues. In circulating blood, both the rate of rise of concentration after contrast agent administration and the rate of decrease after the concentration has reached a peak are characteristically higher than those in new blood vessels. This is because the vascular structure of circulating blood is mature, and accordingly little blood leaks into the surroundings. Ordinarily, blood vessels of circulating blood exhibit characteristically a wider cross-section and higher flow rate than those of new blood vessels. In the present embodiment, regions of circulating blood are identified by exploiting thus the different features of blood vessels of circulating blood and new blood vessels.

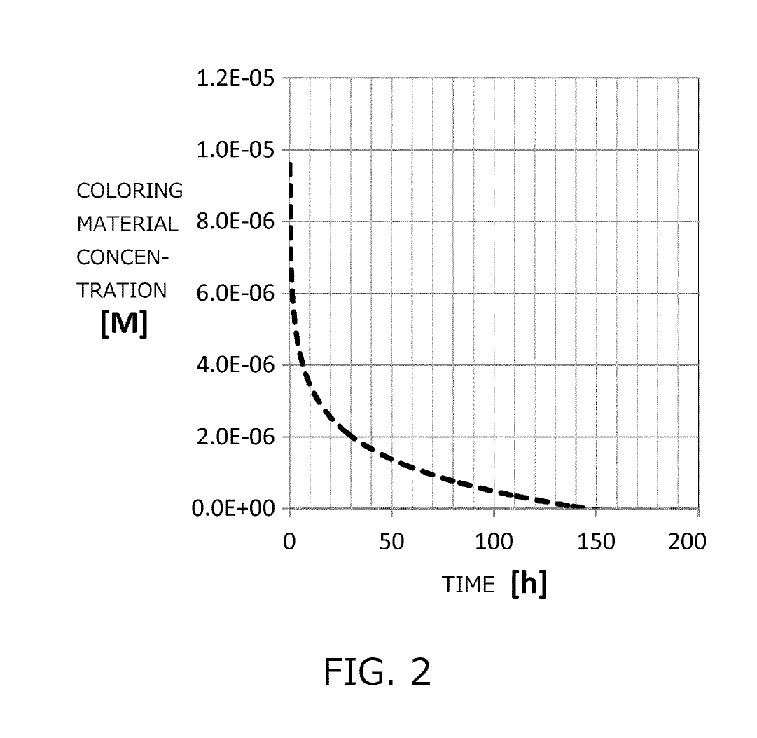

FIG. 2 is a graph illustrating contrast agent concentration change information obtained from a plurality of blood samplings of nude mice to which a contrast agent has been administered. The vertical axis represents the concentration of coloring material in blood, and the horizontal axis denotes the time elapsed since administration. The graph depicts specifically the change of the concentration after administration of contrast agent in an amount of 20 nanomoles of coloring material equivalent, to the caudal vein of the nude mice. As the contrast agent, a material was used in which an indocyanine green derivative, which is a cyanine-based compound, was covalently bonded to polyethylene glycol (PEG), which is a synthetic polymer, having a molecular weight of 20 kDa. The contrast agent will be referred to as “ICG-PEG”. By nature, the contrast agent concentration rises sharply from zero immediately after administration. In FIG. 2, however, that portion has been simplified for the sake of a simpler explanation.

The term “information B” denotes concentration change information of the contrast agent for each unit region (pixel or voxel) of the object, generated over a plurality of times as a result of a plurality of photoacoustic measurements of the object. Information B corresponds to the contrast agent distribution information of the present invention. Information B includes photoacoustic information on both a circulating blood portion and a neovascular portion. In the present embodiment a signal correction target is determined through comparison between information A and information B. Information A need not be acquired simultaneously with information B. Before the photoacoustic measurement, information A pertaining to the living body that is to be measured, or pertaining to another individual similar to the living body to be measured, may be acquired beforehand and stored in a storage device for eventual use. General values of information A for each element, for instance, species, age, sex, body mass and the like, may be stored in the storage device, and be read at a time of use.

In a case where the contrast agent is administered into the circulating blood as one shot, the contrast agent concentration in the circulating blood decreases exponentially due to clearance with the passage of time, as illustrated in FIG. 2. FIG. 2 illustrates a graph from a given point in time at which the contrast agent concentration in the blood has risen after several minutes following administration of the contrast agent. When no signal is acquired within an appropriate lapse of time, therefore, the contrast agent in the circulating blood may in some instances fail to be quantified successfully. For instance, the concentration of the contrast agent is unstable since the contrast agent is distributed unevenly in the circulating blood immediately after administration. On the other hand, once a prolonged period of time has elapsed since administration, the contrast agent concentration in the circulating blood decreases, and becomes readily influenced by the sensitivity and/or measurement variability of the measuring instrument.

The dynamics in the circulating blood after administration to the object vary depending on the type of the contrast agent. In consequence, the range of time that enables detecting the contrast agent concentration stably has to be estimated for each contrast agent. Therefore, a method for estimating a measurement time range according to differences (in particular, differences in retention in blood) depending on the type of the contrast agent will be described next. The contrast agent having been administered into the circulating blood as one shot is deemed to clear according to a first-order rate process. Accordingly, a range over which concentration in blood decreases linearly with respect to a semi-logarithmic axis can be set herein as an appropriate measurement time. By setting thus a threshold value of the measure of linearity for a straight line it becomes possible to set, as an appropriate measurement time, the range within which that threshold value is satisfied.

In the case of FIG. 2, comparatively high linearity (R²=0.81) is exhibited from 15 minutes up to 72 hours after administration, and hence that range can be deemed to be an appropriate measurement time range. On the other hand, linearity drops significantly within 15 minutes of administration, and from 72 hours onwards following administration. Table 1 gives a summary of measurement time ranges for respective contrast agents, with the threshold value of linearity being set arbitrarily (R²≧0.8). Herein the contrast agent is ICG, plus various types of ICG-PEG with respective molecular weights modified in various ways (the values in brackets denote the molecular weight (Da) of PEG).

TABLE 1

| (*1) | (*2) | (*3) | R² |
|---|---|---|---|
| ICG | 0.08~0.5 | 0.8 | 0.84 |
| ICG-PEG (5k) | 0.25~3 | 5 | 0.81 |
| ICG-PEG (10k) | 0.25~6 | 10 | 0.8 |
| ICG-PEG (20k) | 0.25~72 | 20 | 0.81 |
| ICG-PEG (40k) | 0.25~72 | 40 | 0.96 |

(*1) SAMPLE NAME
(*2) TIME [hr]
(*3) MOLECULAR WEIGHT [kDa]

A method for acquiring information A and information B will be explained next as an example of an instance where a plurality of photoacoustic measurements is made in a time-series manner, using light of a plurality of wavelengths, on an object to which a contrast agent has been administered.

The separability of acoustic signals derived from the contrast agent is high in cases where the light absorption characteristic of the contrast agent differs significantly from the light absorption characteristics of other constituents in the object. In those cases, information B for each unit region is obtained by irradiating only light of a wavelength that is absorbed characteristically by the contrast agent, and by performing reconstruction using the obtained photoacoustic signals. In a case where, on the other hand, the contrast agent-derived signal is to be separated from those of other components, light of a plurality of wavelengths is irradiated, and a reconstruction process is performed using photoacoustic signals derived from the respective wavelengths, after which the component corresponding to the contrast agent-derived signal is separated. Isolation targets include, for instance, signals derived from hemoglobin or melanin that are endogenous to the living body.

An instance will be explained next on the separation of hemoglobin signals using light of a plurality of wavelengths. A series of photoacoustic signals measured at a plurality of wavelengths is stored in the storage device in the form of one data set having a same time point allocated thereto. The time point can be set arbitrarily. In a case where, for instance, two-wavelength set measurements are performed every 10 minutes, with one set including a photoacoustic measurement at wavelength λ₁ and a photoacoustic measurement at wavelength λ₂ after 5 seconds since the former photoacoustic measurement, then the time point may be set herein to the point in time of irradiation of light of wavelength λ₁. A data set group acquired at each time point is stored in the storage device.

Spatial information on the light absorber can be generated by performing a reconstruction process for each wavelength, using a time-series data set group. Thereafter, contrast agent-derived signal information can be acquired, for each unit region (voxel, pixel or the like), by removing hemoglobin-derived signals through a computation process in which there is used a plurality of signal values having been acquired at different wavelengths. This computation process is executed by comparing the signal intensities obtained at the two wavelengths, exploiting the differences in light absorption characteristic (wavelength absorption spectrum) between the contrast agent and hemoglobin.

In a more specific example, a method may involve performing measurements at wavelength 1 that allows detecting of the contrast agent and hemoglobin signals, and wavelength 2 that allows detecting of only hemoglobin signals, and subtracting then wavelength 2 from wavelength 1. In another example, a method may involve irradiating light of three wavelengths corresponding to three components, namely oxyhemoglobin, deoxyhemoglobin and a contrast agent, and extracting then the contrast agent signal component from an associated system of equations. In yet another example, a method may rely on analysis techniques, such as spectral unmixing, performed on the respective spectral signals of hemoglobin and a contrast agent. Basic parameters such as light intensity are preferably normalized across wavelengths when performing a computation process at a plurality of wavelengths.

Information B, being time-series contrast agent concentration change information in the unit regions, is obtained by carrying out such a computation process for all the time-series data sets that have been acquired. When saving the information B, the format thereof is immaterial so long as time-series relative differences in blood concentration can be known. For instance, relative values may be used wherein the maximum value of blood concentration is set to 1 (or to a reference value). Alternatively, the information may be approximated by an exponential function or the like on the basis of the acquired data.

Spatial distribution information (hereafter referred to as information C) on hemoglobin having been removed in the computation process may be further stored in a storage unit, to be used in subsequent steps.

(Identification of a Circulating Blood Region)

A method for identifying and extracting unit regions in a circulating blood region will be explained next. Identification methods include firstly a method that involves using information C (hemoglobin distribution information) that reflects vascular structure. Other methods include identification methods that rely on known blood flow measurement techniques such as the Doppler method.

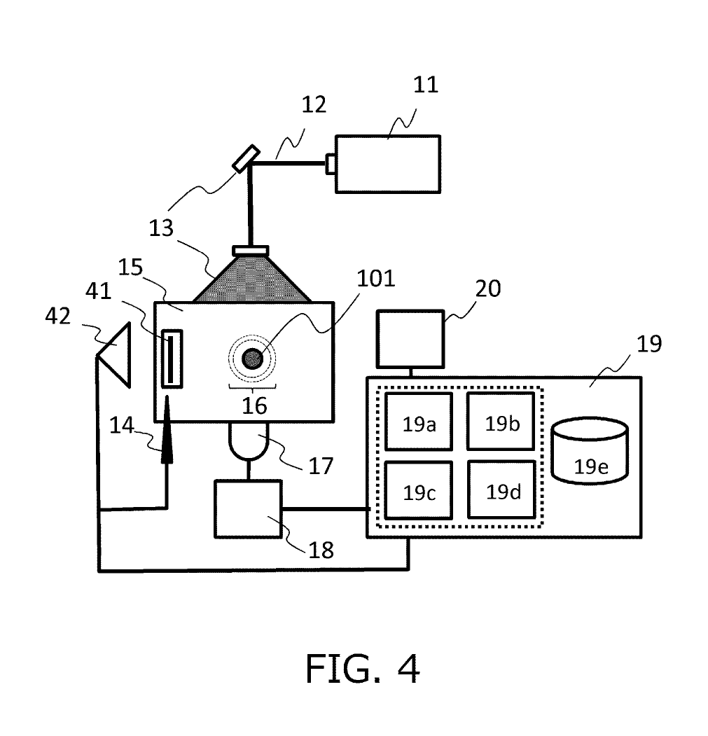

Further identification methods include methods that involve providing an observation unit 41 of the circulating blood in the object and a contrast agent detecting unit 42, as illustrated in FIG. 4, and detecting the contrast agent concentration in synchrony with the start of the acquisition of a time series signal of information B, to acquire information A thereby. The timings of signal acquisition in both information instances are preferably as synchronous as possible. The timings may be worked out on the basis, for instance, of an approximate curve of a time series change of the contrast agent in the circulating blood. The circulating blood observation unit 41 selects and observes an arbitrary object region, for instance a region including superficial blood vessels. For instance the skin, eyes, ears, carotid arteries and caudal vein are examples of regions that include superficial blood vessels. The contrast agent detecting unit 42 detects the contrast agent in accordance with an optical method, such as a photoacoustic method, a fluorescence method and an absorption method, or a known method that relies, for instance, on radioactive dynamic element.

Further, information A may be acquired by referring to a look-up table (LUT) stored beforehand in the storage module 19e. To create the LUT, the contrast agent is administered to the individual; thereafter, blood is sampled according to a time series, and the change in the concentration of the contrast agent comprised in the circulating blood is recorded. The individual for LUT creation is preferably the same individual as the target individual of photoacoustic measurement, but may be a different individual. Statistical values based on time series information of a plurality of individuals may also be used herein. There may be created a table according to age, sex, body mass, lineage and the like. By further approximating the LUT in the form, for instance, of a regression function of time, it becomes possible to compare information B with the change of the concentration of contrast agent of at an arbitrary time. An optical method such as a photoacoustic method, a fluorescence method, and an absorption method or a known method that relies, for instance, on radioactive dynamic element can be used to quantify the contrast agent for creating the LUT. The system can be expected to become simpler, and process times shorter, by using thus an LUT.

(Cross Comparison of Information)

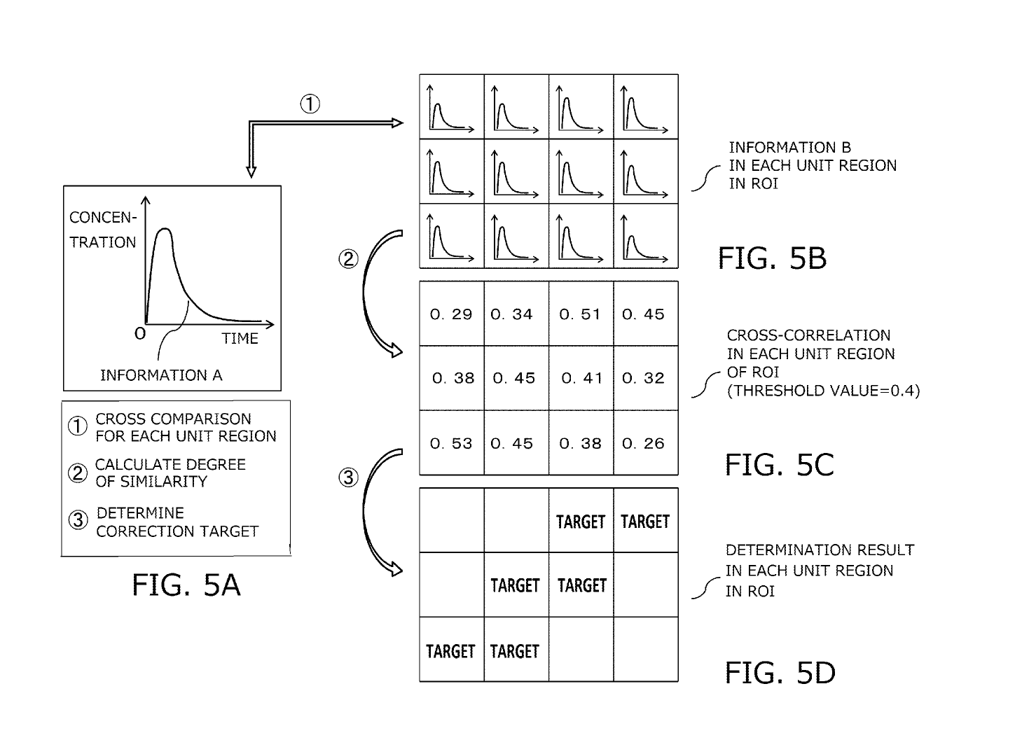

Next, information A and information B are cross-compared for each unit region. In a case where the result of the comparison indicates that the time-series behaviors of both information A and information B are similar for a given unit region, it can be determined that there is a high likelihood that information B for that unit region denotes the contrast agent in the circulating blood. Such a unit region is then extracted, and becomes the target of a correction process that is performed in a subsequent step. The details of the process will be explained with reference to FIG. 5. FIG. 5A is a graph illustrating information A, i.e. the change of the concentration of contrast agent in the circulating blood. FIG. 5B is information B for each unit region.

Firstly, a method for estimating a degree of similarity using a cross-correlation coefficient of information A and information B is described. The cross-correlation coefficient is an index that denotes the degree of similarity of a comparison group.

A specific cross-correlation coefficient can be worked out according to the below-described calculation example. A cross-correlation coefficient r(xy) of information A and information B can be calculated according to Expression (B) below, where X (X1, X2, X3, , , , ) is signal value information (information B in the present invention) at an arbitrary point in time, obtained from blood concentration, and Y (Y1, Y2, Y3, , , , ) is signal value temporal change information (information A in the present invention) obtained from the LUT and corresponding to the arbitrary point in time.

$$\begin{matrix}[ Math . 1 ] & \\ r ( xy ) = \frac{{\sum }_{i = 1}^{N} ( {x}_{i} - \overline{x} ) ( {y}_{i} - \overline{y} )}{\sqrt{{\sum }_{i = 1}^{N} {( {x}_{i} - \overline{x} )}^{2}} \cdot \sqrt{{\sum }_{i = 1}^{N} {( {y}_{i} - \overline{y} )}^{2}}} \\ where , \\ \overline{x} = \frac{1}{N} {\sum }_{i = 1}^{N} {x}_{i} \overline{y} = \frac{1}{N} {\sum }_{i = 1}^{N} {y}_{i} & ( B )\end{matrix}$$

The above expression corresponds to a method that involves calculating differences with respect to the average values of information for X and Y, respectively, to calculate thereby the degree of similarity between X and Y on the basis of a trend for both X and Y. The value is approximated to +1 if there is a trend for both X and Y, to 0 if there is no specific trend, and to −1 if there is a reverse trend.

In a case where, in the calculation of the cross-correlation coefficient, there is a lag in the temporal change information between information X and Y in the relationship of signal acquisition, an operation may be carried out that involves working out the cross-correlation coefficient of information X and Y, after a phase compensation process has been performed beforehand.

By way of example, a value of 0.4 will be set herein as an arbitrary threshold value of the cross-correlation coefficient. That is, unit regions at which information A and information B have a cross-correlation coefficient equal to or higher than 0.4 are determined to be regions of circulating blood.

FIG. 5C illustrates cross-correlation coefficients at each unit region. FIG. 5D illustrates determination results. Whether or not each unit region constitutes a reduction target was determined herein as a two-alternative choice. However, the extent of the reducing process may be adjusted in accordance with the value of the cross-correlation coefficient. That is, the percentage of reduction may be set to be greater as the correlation becomes higher, and to be smaller as the correlation becomes lower.

As another example of cross comparison, there is a method that involves calculating an approximate function A(t) with respect to time, on the basis of information A, and estimating a degree of similarity between A(t) and information B, for each unit region. The estimation method may be, for instance, a technique in which the degree of fitting between information B and A(t) is determined by least-squares. For instance, in a time region where A(t) can be approximated as a linear function, the degree of similarity can be determined by providing a threshold value for linearity (R²). As an example, a value of 0.8 can be set as the threshold value of the linearity (R²). In this case, a unit region having linearity equal to or greater than 0.8 is determined to be a region of circulating blood. In this case as well, a correction process may be performed in which a larger degree of reduction is set as the value of linearity becomes higher.

As another example of cross comparison, there is a method that involves calculating respective regression functions A(t) and B(t) of information A and information B, and working out a degree of similarity by performing a significant difference determination. In a case where the t-test is utilized for significant difference determination, the degree of similarity with a circulating blood signal component can be determined for each data sequence by setting beforehand a threshold value of the p-value. As a guideline, the threshold value of the p-value can be set to be lower than 5%, more preferably lower than 1%. In this case, unit regions having a p-value lower than 1% are determined to be regions of circulating blood. In this case as well, the degree of reduction may be modified in accordance with the p-value.

As yet another example of cross comparison, there is a method that involves calculating and comparing respective variations over time of information A and information B. Specifically, the method involves calculating a respective signal change amount (or slope) within a prescribed time period, and determining a degree of similarity with the circulating blood signal, through detection of significant difference between the two signal change amounts that have been calculated. A threshold value may be provided beforehand as the significant difference coefficient.

The degree of similarity between information A and information B for each unit region can be determined through such cross comparison. A value reduction process (including value elimination) is performed for optical characteristic values of unit regions for which the determination value exceeds a predetermined threshold value and that have been extracted as circulating blood regions. The correction process may be carried out in such a manner that a greater degree of reduction is set as the determination value denoting a degree of similarity becomes larger.

As a result of the correction process of the present embodiment, image data is obtained in which the component derived from circulating blood is reduced, while the portion of new blood vessels corresponding to a tumor is emphasized, and hence useful information can be provided for diagnosis. In other words, images of surrounding tissues such as tumors that include new blood vessels, can be acquired, with good contrast, by using the photoacoustic apparatus according to the present embodiment. Moreover, there is no need for waiting until clearance of the contrast agent in the circulating blood during the photoacoustic measurement; accordingly, measurement times can be shortened while easing the burden to the subject and reducing costs.

Information C (hemoglobin distribution) may be used for unit region extraction. A blood flow information acquisition unit that acquires blood flow information of the object in accordance with the Doppler method may be further provided. As a result, it becomes then possible to identify unit regions that exhibit high correlation for information A and for which signals are obtained that derive from blood vessels. The likelihood of erroneous determination is therefore reduced, and the precision of correction is enhanced as a result.

(Preferred Device Configuration)

The constituent elements of the device will be explained next in detail.

(Light Source)

In a case where the object is a living body, a light source irradiates light of a specific wavelength that is absorbed by specific components contained in the living body (for instance, blood or a light absorber such as a photoacoustic contrast agent). Preferred light sources include pulsed light sources capable of generating pulsed light in the order of several nanoseconds to several hundreds of nanoseconds. A laser is a preferred light source herein. However, a light-emitting diode, a flash lamp or the like may be used as well. For instance, a solid laser, gas laser, coloring material laser or semiconductor laser can be used as the laser. Various effects such as enhancement of light irradiation intensity, widening of the irradiation region and homogenization of the irradiation distribution can be achieved herein through the use of a plurality of light sources and/or a plurality of emission ends.

Preferably, the light source is capable of irradiating light of a plurality of wavelengths, in order to measure differences derived from the wavelength in an optical characteristic value distribution. To that end, there are methods that involve using a plurality of light sources having mutually different lasing wavelengths, and methods that involve using a wavelength-modified laser. Preferred wavelength-modified lasers are herein laser devices that utilize coloring materials that are capable of converting lasing wavelengths, or laser devices that utilize OPOs (Optical Parametric Oscillators).

The wavelength of the irradiation light lies preferably in a region from 700 nm to 1100 nm, within which light is not readily absorbed in vivo. However, a wider wavelength region (for instance, in the range 400 nm to 1600 nm) can be used in cases of measurements that are comparatively close to the surface of the living body. The time width of the light pulses is preferably set to a width such that thermal and stress confinement conditions apply, in order to efficiently confine absorbed energy in the light absorber. The time width ranges typically from about 1 nanosecond to 200 nanoseconds.

(Optical System)

The optical system 13 may utilize any member, so long as light can be guided to the object while being processed to a desired light distribution shape. For instance, optical components such as lenses and mirrors, optical waveguides such as optical fibers, and also light diffuser plates can be used herein.

(Contrast Agent)

In the present description, the term contrast agent denotes a light absorber that is administered externally to the object, mainly for the purpose of improving the contrast (SN ratio) of a photoacoustic signal distribution. Besides light absorbers themselves, the contrast agent may include materials that control in-vivo kinetics. Examples of materials that control in-vivo kinetics include, for instance, serum-derived proteins such as albumin and IgG, and water-soluble synthetic polymers such as polyethylene glycol. Accordingly, the contrast agent in the specification of the present invention encompasses light absorbers themselves, contrast agents in which a light absorber and another material are bonded covalently, and contrast agents in which a light absorber and another material are held by physical interactions.

In the case where the object is a living body, near-infrared light (wavelength from 600 nm to 900 nm) is preferred as the irradiation light, from the viewpoint of safety and biological transmissivity. Accordingly, a material having a light absorption characteristic at least in the near-infrared wavelength region is used as the contrast agent. Examples thereof include, for instance, organic compounds such as cyanine-based compounds (also referred to as cyanine coloring materials) typified by indocyanine green, and inorganic compounds typified by gold or iron oxide.

Preferably, the cyanine-based compound in the present embodiment has a molar extinction coefficient, at an absorption maximum wavelength, of 10⁶ M⁻¹ cm⁻¹ or higher. Examples of structures of the cyanine-based compound in the present embodiment include, for instance, the structures represented by formulas (1) through (4) below.

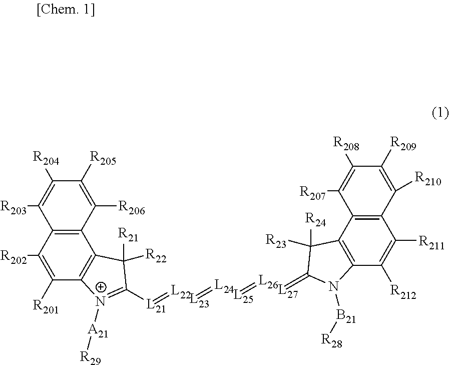

In Formula (1), R₂₀₁ to R₂₁₂ represent, each independently, a hydrogen atom, a halogen atom, SO₃T₂₀₁, PO₃T₂₀₁, a benzene ring, a thiophene ring, a pyridine ring or a linear or branched alkyl group having 1 to 18 carbon atoms, where T₂₀₁ represents any one of a hydrogen atom, a sodium atom and a potassium atom. In Formula (1), R₂₁ to R₂₄ represent, each independently, a hydrogen atom or a linear or branched alkyl group having 1 to 18 carbon atoms. In Formula (1), A₂₁ and B₂₁ represent, each independently, a linear or branched alkylene group having 1 to 18 carbon atoms. In Formula (1), L₂₁ to L₂₇ are each independently CH or CR₂₅, where R₂₅ represents a linear or branched alkyl group having 1 to 18 carbon atoms, a halogen atom, a benzene ring, a pyridine ring, a benzyl group, ST₂₀₂ or a linear or branched alkylene group having 1 to 18 carbon atoms, where T₂₀₂ represents a linear or branched alkyl group having 1 to 18 carbon atoms, a benzene ring or a linear or branched alkylene group having 1 to 18 carbon atoms. In Formula (1), L₂₁ to L₂₇ may form a 4-membered ring to 6-membered ring. In Formula (1), R₂₈ represents any one of —H, —OCH₃, —NH₂, —OH, —CO₂T₂₈, —S(═O)₂OT₂₈, —P(═O)(OT₂₈)₂, —CONH—CH (CO₂T₂₈)—CH₂(C═O)OT₂₈, —CONH—CH (CO₂T₂₈)—CH₂CH₂(C═O)OT₂₈ and —OP(═O)(OT₂₈)₂, where T₂₈ represents any one of a hydrogen atom, a sodium atom and a potassium atom. In Formula (1), R₂₉ represents any one of —H, —OCH₃, —NH₂, —OH, —CO₂T₂₉, —S(═O)₂OT₂₉, —P(═O)(OT₂₉)₂, —CONH—CH(CO₂T₂₉)—CH₂(C═O)OT₂₉, —CONH—CH (CO₂T₂₉)—CH₂CH₂(C═O)OT₂₉, and —OP(═O)(OT₂₉)₂, where T₂₉ represents any one of a hydrogen atom, a sodium atom and a potassium atom.

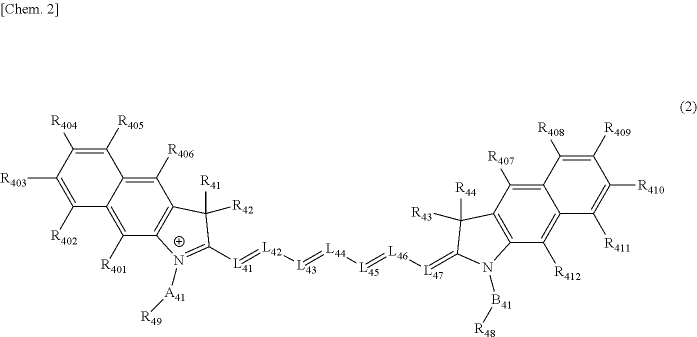

In Formula (2), R₄₀₁ to R₄₁₂ represent, each independently, a hydrogen atom, a halogen atom, SO₃T₄₀₁, PO₃T₄₀₁, a benzene ring, a thiophene ring, a pyridine ring or a linear or branched alkyl group having 1 to 18 carbon atoms, where T₄₀₁ represents any one of a hydrogen atom, a sodium atom and a potassium atom. In Formula (2), R₄₁ to R₄₄ represent, each independently, a hydrogen atom or a linear or branched alkyl group having 1 to 18 carbon atoms. In Formula (2), A₄₁ and B₄₁ represent, each independently, a linear or branched alkylene group having 1 to 18 carbon atoms. In Formula (2), L₄₁ to L₄₇ are each independently CH or CR₄₅, where R₄₅ represents a linear or branched alkyl group having 1 to 18 carbon atoms, a halogen atom, a benzene ring, a pyridine ring, a benzyl group, ST₄₀₂ or a linear or branched alkylene group having 1 to 18 carbon atoms, where T₄₀₂ represents a linear or branched alkyl group having 1 to 18 carbon atoms, a benzene ring or a linear or branched alkylene group having 1 to 18 carbon atoms. In Formula (2), L₄₁ to L₄₇ may form a 4-membered ring to 6-membered ring. In Formula (2), R₄₈ represents any one of —H, —OCH₃, —NH₂, —OH, —CO₂T₄₈, —S(═O)₂OT₄₈, —P(═O)(OT₄₈)₂, —CONH—CH (CO₂T₄₈)—CH₂(C═O)OT₄₈, —CONH—CH (CO₂T₄₈)—CH₂CH₂(C═O)OT₄₈, and —OP(═O)(OT₄₈)₂, where T₄₈ represents any one of a hydrogen atom, a sodium atom and a potassium atom. In Formula (2), R₄₉ represents any one of —H, —OCH₃, —NH₂, —OH, —CO₂T₄₉, —S(═O)₂OT₄₉, —P(═O)(OT₄₉)₂, —CONH—CH(CO₂T₄₉)—CH₂(C═O)OT₄₉, —CONH—CH (CO₂T₄₉)—CH₂CH₂(C═O)OT₄₉, and —OP(═O)(OT₄₉)₂, where T₄₉ represents any one of a hydrogen atom, a sodium atom and a potassium atom.

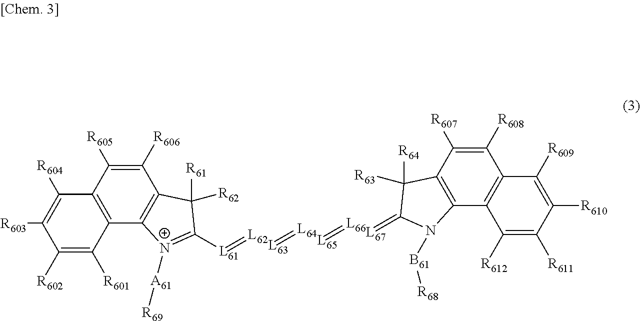

In Formula (3), R₆₀₁ to R₆₁₂ represent, each independently, a hydrogen atom, a halogen atom, SO₃T₆₀₁, PO₃T₆₀₁, a benzene ring, a thiophene ring, a pyridine ring or a linear or branched alkyl group having 1 to 18 carbon atoms, where T₆₀₁ represents any one of a hydrogen atom, a sodium atom and a potassium atom. In Formula (3), R₆₁ to R₆₄ represent, each independently, a hydrogen atom or a linear or branched alkyl group having 1 to 18 carbon atoms. In Formula (3), A₆₁ and B₆₁ represent, each independently, a linear or branched alkylene group having 1 to 18 carbon atoms. In Formula (3), L₆₁ to L₆₇ are each independently CH or CR₆₅, where R₆₅ represents a linear or branched alkyl group having 1 to 18 carbon atoms, a halogen atom, a benzene ring, a pyridine ring, a benzyl group, ST₆₀₂ or a linear or branched alkylene group having 1 to 18 carbon atoms, where T₆₀₂ represents a linear or branched alkyl group having 1 to 18 carbon atoms, a benzene ring or a linear or branched alkylene group having 1 to 18 carbon atoms. In Formula (3), L₆₁ to L₆₇ may form a 4-membered ring to 6-membered ring. In Formula (3), R₆₈ represents any one of —H, —OCH₃, —NH₂, —OH, —CO₂T₆₈, —S(═O)₂OT₆₈s, —P(═O)(OT₆s)₂, —CONH—CH (CO₂T₆₈)—CH₂(C═O)OT₆₈, —CONH—CH (CO₂T₆₈)—CH₂CH₂(C═O)OT₆₈, and —OP(═O)(OT₆₈)₂, where T₆₈ represents any one of a hydrogen atom, a sodium atom and a potassium atom. In Formula (3), R₆₉ represents any one of —H, —OCH₃, —NH₂, —OH, —CO₂T₆₉, —S(═O)₂OT₆₉, —P(═O)(OT₆₉)₂, —CONH—CH(CO₂T₆₉)—CH₂(C═O)OT₆₉, —CONH—CH (CO₂T₆₉)—CH₂CH₂(C═O)OT₆₉, and —OP(═O)(OT₆₉)₂,

The T₆₉ represents any one of a hydrogen atom, a sodium atom and a potassium atom.

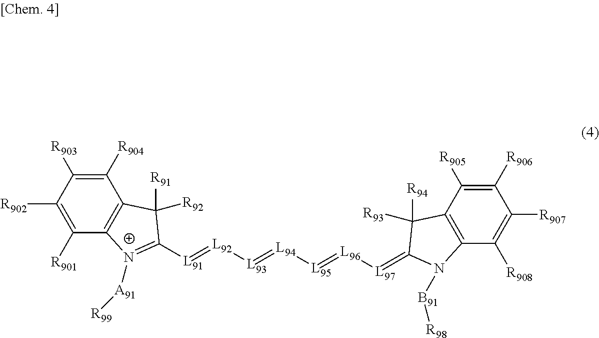

In Formula (4), R₉₀₁ to R₉₀₈ represent, each independently, a hydrogen atom, a halogen atom, SO₃T₉₀₁, PO₃T₉₀₁, a benzene ring, a thiophene ring, a pyridine ring or a linear or branched alkyl group having 1 to 18 carbon atoms, where T₉₀₁ represents any one of a hydrogen atom, a sodium atom and a potassium atom. In Formula (4), R₉₁ to R₉₄ represent, each independently, a hydrogen atom or a linear or branched alkyl group having 1 to 18 carbon atoms. In Formula (4), A₉₁ and B₉₁ represent, each independently, a linear or branched alkylene group having 1 to 18 carbon atoms. In Formula (4), L₉₁ to L₉₇ are each independently CH or CR₉₅, where R₉₅ represents a linear or branched alkyl group having 1 to 18 carbon atoms, a halogen atom, a benzene ring, a pyridine ring, a benzyl group, ST₉₀₂ or a linear or branched alkylene group having 1 to 18 carbon atoms, where T₉₀₂ represents a linear or branched alkyl group having 1 to 18 carbon atoms, a benzene ring or a linear or branched alkylene group having 1 to 18 carbon atoms. In Formula (4), L₉₁ to L₉₇ may form a 4-membered ring to 6-membered ring. In Formula (4), R₉₈ represents any one of —H, —OCH₃, —NH₂, —OH, —CO₂T₉₈, —S(═O)₂OT₉₈, —P(═O)(OT₉₈)₂, —CONH—CH (CO₂T₉₈)—CH₂(C═O)OT₉₈, —CONH—CH (CO₂T₉₈)—CH₂CH₂(C═O)OT₉₈, and —OP(═O)(OT₉₈)₂, where T₉₈ represents any one of a hydrogen atom, a sodium atom and a potassium atom. In Formula (4), R₉₉ represents any one of —H, —OCH₃, —NH₂, —OH, —CO₂T₉₉, —S(═O)₂OT₉₉, —P(═O)(OT₉₉)₂, —CONH—CH(CO₂T₉₉)—CH₂(C═O)OT₉₉, —CONH—CH (CO₂T₉₉)—CH₂CH₂(C═O)OT₉₉, and —OP(═O)(OT₉₉)₂, where T₉₉ represents any one of a hydrogen atom, a sodium atom and a potassium atom.

Examples of the cyanine-based compound in the present embodiment include indocyanine green, SF-64 having a benzotricarbocyanine structure and represented by Chemical formula 1, and compounds represented by Chemical formulas (i) through (v).

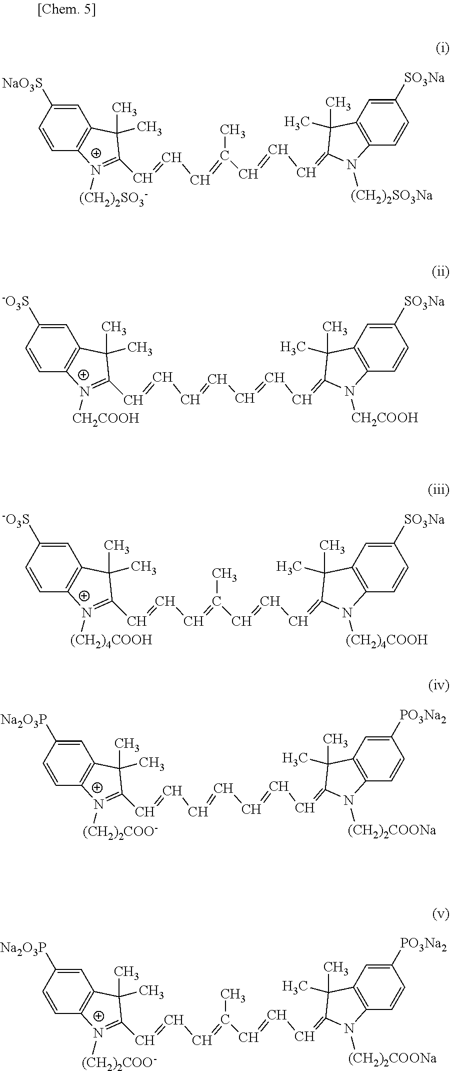

The aromatic rings in the above cyanine-based compounds may be substituted with a sulfonate group, a carboxyl group or a phosphate group. Sulfonate groups, carboxyl groups and phosphate group may also be introduced at portions other than the aromatic rings.

(Contrast Agent Administering Unit)

The contrast agent administering unit 14 administers the contrast agent from outside the object thereinto. The time at which the administration operation by the contrast agent administering unit 14 is completed constitutes a starting point of execution of a below-described discrimination process of signals derived from circulating blood. The contrast agent administering unit may be configured arbitrarily, so long as the contrast agent can be administered to the object via a vein or the like. For instance existing injection systems, injectors and so forth can be used herein. The contrast agent administering unit transmits the time at which the administration operation is completed to a below-described signal processing unit. The administration method is not particularly limited, and may be, for instance, bolus administration.

(Acoustic Wave Detecting Unit)

The acoustic wave detecting unit 17 is a probe provided with a detection element that detects acoustic waves propagating from the object and that converts the detected acoustic waves to an analog electrical signal. Examples of the detection element that can be used herein include, for instance, elements that rely on piezoelectric phenomena, elements relying on light resonance, or elements that rely on changes in capacitance. Preferably, a probe is used in which a plurality of detection elements is disposed uni-dimensionally or two-dimensionally. As a result, it becomes possible to detect acoustic waves at a plurality of sites simultaneously, which contributes therefore to shortening detection times and reducing the influence of object vibration. Preferably, the device is provided with a driving means such as a stepping motor or a stage, for moving the acoustic wave detecting unit 17 to an arbitrary position. The object can be detected as a result in various directions; hence, the amount of information that is used in reconstruction can be enhanced and image quality improved.

(Signal Collecting Unit)

The signal collecting unit 18 performs an amplification process and a digital conversion process on the analog electrical signal outputted by the acoustic wave detecting unit. The signal collecting unit is typically made up of an amplifier, an A/D converter, an FPGA (Field Programmable Gate Array) chip or the like. The digital electrical signal outputted by the signal collecting unit is transmitted to the signal processing unit, and is stored in a storage module 19e which is a storage means.

(Signal Processing Unit)

The signal processing unit 19 performs image reconstruction using the digital signal stored in the storage module 19e, to image the contrast agent distribution information. At this time, the signal component derived from contrast agent in the circulating blood is deleted or reduced. As a result, the visualization performance of the contrast agent that migrates from the circulating blood to the surrounding tissue (tumor or the like) and accumulates therein is enhanced, also under measurement conditions in which the contrast agent stays in the circulating blood.

A data processing device, for instance a PC or workstation, provided with a processor and that operates according to software is preferable herein as the signal processing unit. The software includes a signal processing module 19a, a signal discrimination module 19b, a signal correction module 19c, and a signal imaging module 19d.

The signal processing module 19a reads a plurality of time-series photoacoustic signals from the storage module 19e, separates an absorber signal, for instance of endogenous hemoglobin, and acquires temporal change information of a signal component derived from the contrast agent. To separate the signals a computation process is performed using the results of the photoacoustic measurement according to a plurality of wavelengths. For instance, there is a method that involves removing hemoglobin signals through subtraction or the like in a two-wavelength measurement. Another method involves performing a three-component computation of oxy-deoxyhemoglobin and a contrast agent, in a three-wavelength measurement. Yet another method involves computing a fraction of the contrast agent from curve fitting using a least-squares method or the like, in a multi-wavelength measurement.

The signal discrimination module 19b compares the measured change of the concentration of contrast agent (information B) and the change of the concentration of contrast agent in the circulating blood (information A), for each unit region. Then, based on the methods described above, the signal discrimination module 19b measures the degree of similarity between information A and information B, and determines whether or not the unit region is a circulating blood-derived unit region that is to be corrected. Alternatively, the signal discrimination module 19b may work out a correction degree in accordance with a circulating blood region likelihood. Further, the signal discrimination module 19b performs a reduction process, including deletion, on the contrast agent signal component derived from circulating blood, to generate a corrected detection signal.

The signal imaging module 19d performs image reconstruction using the corrected signal, to generate image data of the interior of the object. Methods that are ordinarily used in tomographic technology can be used herein as an image reconstruction algorithm. Examples thereof include, for instance, reverse projection in the time domain or the Fourier domain, Fourier transform, universal back projection, filtered back projection, deconvolution, iterative reconstruction, inverse problem analysis and the like. Images can be generated, even without image reconstruction, by acquiring photoacoustic waves through scanning of an arbitrary region using a focusing-type ultrasonic probe in the acoustic wave detecting unit.

The timing of image reconstruction may be subsequent to the process by the signal correction module 19c as described above. The spatial distribution information of light absorber may be acquired through execution of image reconstruction at the signal imaging module 19d first, followed subsequently by execution of the processes by the signal processing module 19a, the signal discrimination module 19b and the signal correction module 19c, for the signals of each unit region. Preferably, the signal processing unit 19 is interlocked with the contrast agent administering unit 14, to synchronize thereby contrast agent administration, acoustic wave acquisition, and measurement of the blood concentration change of the contrast agent.

The above module division is an example, and the signal processing unit may adopt any form, so long as the signal processing unit can execute the steps that are carried out in each module. Specifically, the signal processing unit may be configured to discriminate, by software or by way of a process circuit, a contrast agent signal component derived from circulating blood on the basis of a digital signal outputted by the signal collecting unit, and to perform a correction process such as a reduction process.

(Display Device)

The display device 20 displays the image data that is outputted by the signal processing unit. A liquid crystal display, a plasma display or a CRT can be used herein. The display device may be provided separately from the main body of the device of the present invention.

(Object Information Acquisition Method)

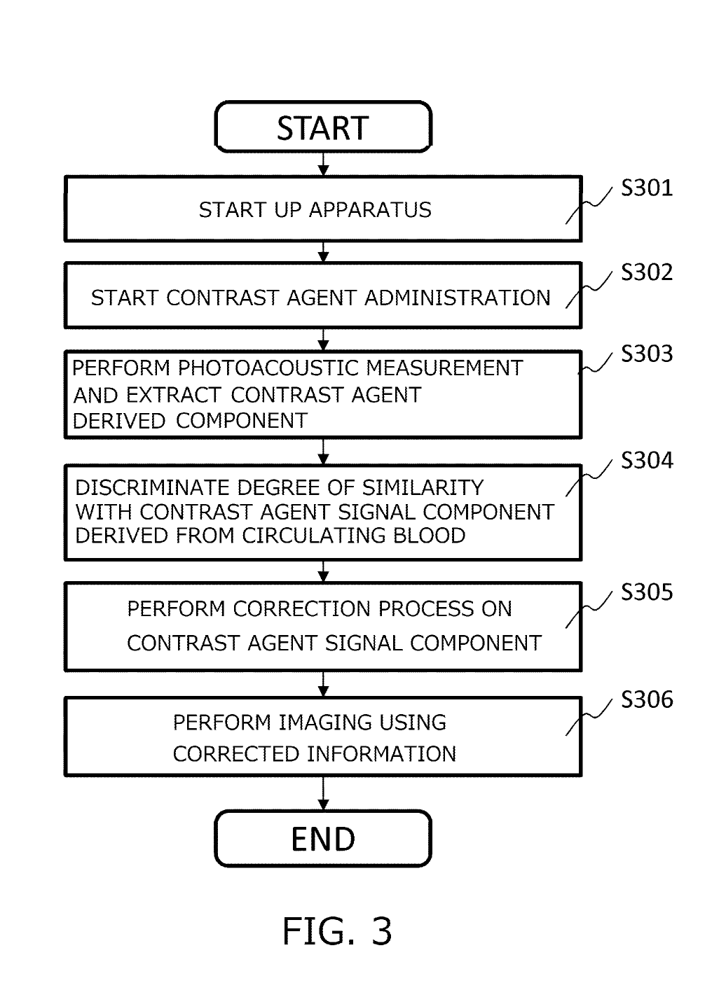

The process executed by the signal processing unit 19 will be explained next with reference to the flowchart of FIG. 3.

Process (1) (Step S301): A Step of Starting Up the Device

Firstly, the settings of the object are applied and the device is started up.

Process (2) (Step S302): A Step of Administering the Contrast Agent

The contrast agent administering unit 14 administers the contrast agent, containing an absorber, into the object.

Process (3) (Step S303): A Step of Photoacoustic Measurement and Contrast Agent-Derived Component Extraction

In the present step, the device performs a plurality of time-series photoacoustic measurements, at predetermined timings, to obtain photoacoustic signals. The signal processing unit acquires a time-series change of the concentration of contrast agent (information B) for each unit region, using the photoacoustic signal. The method for extracting the contrast agent-derived component at this time has been described above.

The present step may include a step of initiating a time count for synchronization. The time count is performed in a case where the contrast agent concentration change information in the circulating blood (information A) is not acquired simultaneously with the photoacoustic signals. This applies specifically to an instance where the contrast agent concentration change information in the circulating blood is referenced from an LUT, or an instance where the signal in the circulating blood is acquired from a segment (for instance, a superficial blood vessel of the object) that is different from the measurement target within the object. In a case where, for instance, a general injection system is used in an angiographic system for X-ray CT, a trigger signal may be sent to the photoacoustic apparatus, and the time count initiated, once administration of contrast agent is completed. In a case where signal extraction is performed using a reconstructed image, the reconstruction process is carried out after the photoacoustic measurement in the present step and before the extraction process.

Process (4) (Step S304): A Step of Discriminating a Degree of Similarity with a Contrast Agent Signal Component Derived from Circulating Blood

As a premise of the present step, the signal processing unit has acquired information A pertaining to the circulating blood in accordance with a method such as referring to an LUT. Next, the signal processing unit compares the time-series change of the concentration of contrast agent obtained in the previous step with the referenced information A, to discriminate thereby the degree of similarity between the foregoing two information instances, and extract unit regions to be corrected.

Other methods for acquiring information on the change of signals derived from circulating blood involve acquiring a contrast agent-derived signal from superficial blood vessels, as illustrated in FIG. 4, to quantify thereby the contrast agent in a time-series fashion. Yet other methods involve designating a specific portion out of a region of interest, and acquiring information on the change of signals in the circulating blood in accordance with an optical method that relies on photoacoustics, fluorescence or the like, or a method in which radioactive elements are utilized. A hemoglobin distribution, blood flow information and so forth may be used concomitantly when employing such methods.

Process (5) (Step S305): A Step of Performing a Correction Process on a Contrast Agent Signal Component

The signal processing unit performs a process of correcting the data of unit regions that have been determined, in the previous step, to be unit regions to be corrected. Examples of correction methods include, for instance, methods that involve setting to 0 the value of data to be corrected. A method may be employed that involves creating binary mask data in which 0 is allocated to data for which a high degree of similarity has been determined and 1 is allocated to data for which a low degree of similarity has been determined, and superposing thereupon the mask data on the object information. Further, correction coefficients may be determined using a table or numerical expression, in accordance with the degree of similarity between information A and information B.

The purport of the correction process is not limited to a signal reduction process. To display object information on the display device, for instance, a method can be applied that involves modifying the tone and display color of regions to be corrected, to thereby visually separate and display the regions.

Process (6) (Step S306): A Step of Performing Imaging Using Corrected Information

The signal processing unit converts the object signal corrected in the previous step to image data, and displays the image data on the display device. Herein MIP display, in which there is projected a maximum brightness value in a direction in which all signal values can be made into images, is suitable in the case of three-dimensional image data. Other display methods may however be used.

The above process flow allows imaging, with sufficient contrast, photoacoustic waves that are generated from a contrast agent that is distributed in new blood vessels and/or extravascularly, for any time range over which the contrast agent that has been administered into the living body is present in the circulating blood. As a result, the distribution of contrast agent that permeates through blood vessels and new blood vessels and reaches surrounding tissue such as a tumor can be accurately displayed, and information that is useful for diagnosis can be provided.

### Embodiment 2

The present embodiment is identical to Embodiment 1 as regards the feature of generating image data in which the signal intensity of portions having been determined as circulating blood regions is reduced or expunged. In the present embodiment, however, there is generated light absorber distribution information, being photoacoustic image data derived from a light absorber other than a contrast agent, instead of, or along with, a contrast agent image.

Examples of light absorbers other than contrast agents include firstly information pertaining to blood hemoglobin. For instance, a hemoglobin distribution image in which profuse tumors or new blood vessels in deep portions of the object are emphasized is obtained through reduction of the signal intensity of portions corresponding to a circulating blood region, on the basis of a hemoglobin distribution that is obtained as the information C. The same applies to an oxygen saturation distribution image obtained by comparing the intensities of oxyhemoglobin and deoxyhemoglobin. Further, high-contrast image data in which the influence of circulating blood is reduced can be generated also for distributions of substances other than hemoglobin, for instance a glucose concentration distribution.

The present invention allows acquiring images of surrounding tissue such as a tumor, with good contrast, in photoacoustic tomography where a contrast agent is used.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-064323, filed on Mar. 26, 2015, which is hereby incorporated by reference herein in its entirety.

1. A photoacoustic apparatus, comprising:
a light source;
a detecting unit that detects photoacoustic waves generated upon irradiation of light, from the light source, onto an object that has first blood vessels in which circulating blood circulates and second blood vessels having a structure different from that of the first blood vessels, the object containing a contrast agent in the first and second blood vessels; and
a signal processing unit that generates contrast agent distribution information by working out a concentration of the contrast agent in each unit region within the object using the photoacoustic waves, and that acquires contrast agent concentration change information denoting the change with time of the concentration of the contrast agent in the circulating blood,
wherein the signal processing unit:
generates the contrast agent distribution information a plurality of times in response to a plurality of light irradiations from the light source;
acquires the position of the first blood vessels on the basis of a time-series change of the contrast agent distribution information having been generated a plurality of times, and the contrast agent concentration change information; and
performs correction of lowering the concentration of the contrast agent at the position of the first blood vessels on the basis of the contrast agent distribution information.

2. The photoacoustic apparatus according to claim 1, wherein the signal processing unit calculates, for each unit region in the object, a time-series change of the concentration of the contrast agent and a degree of similarity with the contrast agent concentration change information.

3. The photoacoustic apparatus according to claim 2, wherein the signal processing unit determines that the unit region corresponds to the first blood vessels when the degree of similarity is greater than a predetermined value.

4. The photoacoustic apparatus according to claim 2, wherein the signal processing unit sets a greater extent of correction of lowering the concentration of the contrast agent in the unit region as the degree of similarity is increased.

5. The photoacoustic apparatus according to claim 1, further comprising a storage device that stores the contrast agent concentration change information,
wherein the signal processing unit acquires the contrast agent concentration change information by referring to the storage device.

6. The photoacoustic apparatus according to claim 1, wherein the signal processing unit calculates the contrast agent concentration change information on the basis of the contrast agent distribution information.

7. The photoacoustic apparatus according to claim 6,
wherein the light source can radiate light of a plurality of wavelengths, and
the signal processing unit:
generates a plurality of items of the contrast agent distribution information for each of the wavelengths;
separates a signal component derived from the contrast agent and a signal component derived from a substance other than the contrast agent within the object using the plurality of items of contrast agent distribution information; and
calculates the contrast agent concentration change information on the basis of the signal component derived from the contrast agent.

8. The photoacoustic apparatus according to claim 7,
wherein the substance other than the contrast agent is hemoglobin, and
the photoacoustic apparatus further comprises a storage device that stores the signal component derived from the hemoglobin.

9. The photoacoustic apparatus according to claim 8, wherein the signal processing unit uses information pertaining to the signal component derived from the hemoglobin, when acquiring the position of the first blood vessels.

10. The photoacoustic apparatus according to claim 1, further comprising a contrast agent detecting unit that detects the contrast agent concentration in the object,
wherein the signal processing unit acquires the contrast agent concentration change information using an output of the contrast agent detecting unit.

11. The photoacoustic apparatus according to claim 10, wherein the contrast agent detecting unit detects the contrast agent concentration in synchrony with detection of the photoacoustic waves by the detecting unit.

12. The photoacoustic apparatus according to claim 2, wherein the signal processing unit calculates the degree of similarity using a cross-correlation coefficient.

13. The photoacoustic apparatus according to claim 2, wherein the signal processing unit calculates the degree of similarity by fitting the time-series change of the concentration of the contrast agent to an approximate function of the contrast agent concentration change information.

14. The photoacoustic apparatus according to claim 2, wherein the signal processing unit calculates the degree of similarity by performing significant difference determination of respective regression functions of the contrast agent concentration change information and of the time-series change of the concentration of the contrast agent.

15. The photoacoustic apparatus according to claim 1, further comprising a blood flow information acquisition unit that acquires blood flow information of the object in accordance with a Doppler method, wherein the signal processing unit uses the blood flow information when acquiring the position of the first blood vessels.

16. The photoacoustic apparatus according to claim 1, wherein the second blood vessels are new blood vessels at the periphery of a tumor within the object.

17. The photoacoustic apparatus according to claim 1, wherein the second blood vessels have a structure in which blood permeates into surrounding tissue more readily than in the case of the first blood vessels.

18. A photoacoustic apparatus, comprising:
a light source;
a detecting unit that detects photoacoustic waves generated upon irradiation of light, from the light source, onto an object that has first blood vessels in which circulating blood circulates and second blood vessels having a structure different from that of the first blood vessels, the object containing a contrast agent in the first and second blood vessels; and
a signal processing unit that generates light absorber distribution information by working out a concentration of a light absorber for each unit region within the object using the photoacoustic waves, generates contrast agent distribution information within the object using the light absorber distribution information, and acquires contrast agent concentration change information that denotes a change with time of the concentration of the contrast agent in the circulating blood,
wherein the signal processing unit:
generates the contrast agent distribution information a plurality of times in response to a plurality of light irradiations from the light source;
acquires the position of the first blood vessels on the basis of a time-series change of the contrast agent distribution information having been generated a plurality of times, and the contrast agent concentration change information; and
performs correction of lowering the concentration of the light absorber at the position of the first blood vessels, on the basis of the light absorber distribution information.
